(19) United States
(12) Patent Application Publication
(10) Pub. No.: US 2018/0353429 A1
(43) Pub. Date: Dec. 13, 2018

(54) PRODUCT COMPRISING HYDROLIZED COLLAGEN AND AT LEAST ONE AMORPHOUS MICRONIZED DRUG, PROCESS FOR THE PREPARATION THEREOF AND RELATED USES IN MEDICAL FIELD

(71) Applicants: S.B.M. S.r.l., Roma (IT); 3M&F Consulting S.r.l.s., Roma (IT)

(72) Inventors: Bruno SILVESTRINI, Roma (IT); Vincenzo MOLLACE, Roma (IT)

(21) Appl. No.: 15/781,155

(22) PCT Filed: Dec. 2, 2016
(86) PCT No.: PCT/IT2016/000284

§ 371 Date: Jun. 4, 2018

(30) Foreign Application Priority Data
Dec. 3, 2015 (IT) 102015000079955

(51) Int. Cl. A61K 9/14; A61K 47/42; A61K 31/616; A61K 31/573; A61K 31/192; A61K 31/18; A61K 9/00; A61K 9/48

(52) U.S. Cl. CPC A61K 9/146; A61K 47/42; A61K 31/616; A61K 31/573; A61K 9/4866; A61K 31/18; A61K 9/0014; A61K 9/006; A61K 31/192

(57) ABSTRACT

The present invention relates to a product consisting of a conglomerate of collagen, in the form of the partly hydroyzed industrial derivative thereof called gelatine, and an amorphous micronized drug, characterized by a high bioavailability of the micronized active ingredient and a safety of use of the same deriving from the protective action of collagen against the harmful effects of contact with the active ingredient.

The present invention relates to a product comprising collagen and at least one amorphous micronized drug, a process for the preparation thereof and related uses in medical field.

In greater detail, the invention relates to a product, consisting of a conglomerate of collagen and an amorphous micronized drug, produced by means of a new co-grinding process. The product of the invention is characterized by a high bioavailability of the active micronized pharmaceutical ingredient and safety of the use thereof by virtue of the protective action of the collagen against the harmful effects of contact of the active ingredient.

It is well known that the oral administration of NSAIDs carries a considerable risk of gastrointestinal erosions, haemorrhages and ulcers (Baigent et al., 2009; La García Rodríguez and Jick, 1994; Huang et al., 2011; US Preventive Services Task Force, 2009; Sreenivasa et al., 2012). This risk is to a large extent tied to the erosive contact action of NSAIDs on the gastrointestinal mucosa, which is independent of the systemic erosive action deriving from the inhibition of prostaglandin synthesis (Cioli et al., 1979). Sublingual administration enables NSAIDs to be introduced directly into the bloodstream, thus avoiding the erosive contact action on the gastrointestinal mucosa (Silvestrini and Bonanomi, 2008, 2009). However, sublingual administration carries the risk of transferring the erosive contact action of NSAIDs from the gastrointestinal to the sublingual mucosa.

Collagen, here referring to its partially hydroyzed industrial product called gelatine, is well known for its protective action against contact erosions and ulcers caused by NSAIDs (Castro et al., 2007; Castro et al., 2010; Dingding Chen and Lizhong Gao, 2007; Guangli Mu and Zingfu Ma, 2011; Zhou Xin et al., 2011). This protection is provided also against hydrochloric acid, which contributes to the erosive action of NSAIDs on the stomach (Silvestrini, 2015). Moreover, collagen is considered a safe ingredient, largely used in the food and pharmaceutical fields as is or in the form of an excipient, capsules and devices (Schreiber and Gareis, 2007).

Micronization and amorphization are techniques that are widely used to improve the bioavailability of drugs by facilitating their dissolution and absorption. The former can be achieved both by relying on traditional grinding and compression techniques, and with more modern methods, such as RESS (Rapid Expansion of Supercritical Solutions), SAS (Supercritical Anti-Solvent) and PGSS (Particles from Gas Saturated Solutions) (Cooper and Voelker, 2012; Joshi, 2011; Voelker and Hammer, 2012). Amorphization relies on various methods, such as melting followed by solidification by means of rapid cooling, the evaporation of solutes and lyophilization (Newman et al., 2012; Newman et aL, 2015). Co-grinding of a drug associated with a carrier, consisting of cyclodextrins and other polymers or macromolecules, has the advantage of obtaining the micronization and amorphization of a drug by means of a single process (Carli, 1987; Carli et aL, 2013; Gupta et al., 2003).

Although, as stated above, the properties of collagen and micronization and amorphization techniques are well known, to date it has not been possible to use collagen, in the form of the partly hydroyzed industrial derivative called gelatine, as a carrier in a co-grinding process, in the place of cyclodextrins, polymers and macromolecules, in order to simultaneously improve the local tolerability and bioavailability of drugs. The technical problem that has thus far precluded the above-mentioned use of collagen consists in the glue effect of collagen itself, an effect that manifests itself rapidly during the co-grinding process, preventing it from being completed.

The above-described technical problem is not overcome even by patent U.S. Pat. No. 6,136,336, which concerns formulations of amorphous JM216 (bis-acetato-ammine-dichloro-cyclohexylamine-platinum (IV)) obtained by co-grinding with β-cyclodextrin or other polymers, such as, for example, gelatine, polyvinylpyrrolidinone (PVP) and hydroxypropylmethyl cellulose. According to this patent the drug JM216 amorphized with β-cyclodextrin or with the other polymers mentioned above shows greater water solubility. However, the patent does not describe how it is possible to obtain amorphization of the drug while avoiding the technical problem of the glue effect that occurs during co-grinding with collagen in the form of the industrial product thereof called gelatine. Furthermore, the patent seems to obtain a formulation that is more water soluble and hence not suited for the purpose of obtaining a greater bioavailability through the sublingual mucosa or gastric mucosa.

Based on what has been illustrated above, there is thus an evident need to be able to have new drugs that overcome the disadvantages of the drugs and formulations known up to now.

According to the present invention, it is has now been found that by first reducing the size of the solid particles of the drug until micronization of the same it is possible to achieve amorphization of the drug by co-grinding with collagen, in the form of the industrial product thereof called gelatine.

More precisely, the process of co-grinding according to the present invention envisages grinding a mixture in the form of a dry powder consisting of at least one micronized drug (or pharmaceutical active ingredient) and collagen in the form of the industrial product thereof called gelatine.

The drug usable in the process of the present invention is represented by any drug in solid crystalline form, preferably insoluble or poorly soluble in water, though water-soluble drugs can also be used.

Insofar as the collagen is concerned, this term refers here to the partly hydroyzed industrial derivative thereof called gelatine. It can be mammalian, chicken or fish collagen, for example with a Bloom degree comprised between 0 and 300.

The ratio between collagen and drug can range from 1 to 1 to 1 to 20 and beyond, such as 1 to 30.

As said above, the drug is first micronized in the form of particles with a diameter preferably not exceeding 40 microns. This step, combined with the rapidity with which collagen has demonstrated to be capable of incorporating and stabilizing the amorphous particles, makes it possible to complete the amorphization of the drug before the glue effect prevents it.

The completion of the amorphization process can be promptly verified by optical microscopy. It provides a visual representation of the progressive disappearance of the microcrystalline particles, which are replaced by their amorphous counterpart incorporated into the collagen. Alternatively, the pattern of the amorphization process can be verified by differential scanning calorimetry (DSC), which reveals the disappearance of the transition peak corresponding to the deconstruction of the crystalline scaffold. That amorphization has taken place can moreover be verified with other analytic methods, such as Raman spectroscopy, which relies on an indicator of the diffraction peak corresponding to the crystalline state of a drug.

Through the process of the present invention it is therefore possible to obtain a new product consisting in a combination, or rather a conglomerate, of collagen and at least one amorphous micronized drug. Thanks to the structure of the conglomerate obtainable through the co-grinding process of the invention, the product of the present invention is distinguished by a high bioavailability, as a consequence of an increased liposolubility of the active ingredient obtained thanks to the amorphization of the micronized active ingredient, and a safety of use (or local tolerability) deriving from the protective action of the collagen against the harmful effects of contact. Furthermore, the product of the present invention can be easily produced industrially in the form of pharmaceutical formulations for therapeutic use, both systemic—sublingually and orally—and topical use on mucosa and skin surfaces.

The process of preparation of the present invention has been successfully tested on various drugs, such as acetylsalicylic acid, ibuprofen, aceclofenac, indometacin, ketoprofene, naproxene, ursodeoxycholic acid, carvedilol, dihydroergotamine, furosemide, quinapril and valproic acid; in a more general sense, the preparation process is applicable to any drug susceptible of amorphization using the traditional co-grinding technique (Barzegar-Jalalia et al., 2010; Gohel, 2000; Serajuddin, 1999; Vadher et al., 2009; Watanabe et al., 2002; Wongmekiat et al., 2006).

Therefore, the specific subject matter of the present invention relates to a product, or composition product, comprising or consisting of at least one amorphous micronized drug (or pharmaceutical active ingredient) and collagen, the latter in the form of the partly hydroyzed industrial product thereof called gelatine, said product being obtainable through amorphization by co-grinding at least one crystalline micronized drug in a powder mixture with collagen.

In technical language, the product of the invention is also called a composition product because it is a new product obtained by combining a number of components which, in the specific case of the present invention, are at least one drug and collagen, using a suitable process of preparation. Therefore, the product of the invention can also be designated as a composition product.

The ratio by weight between said at least one drug and the collagen can range from 1:1 to 1:30, preferably from 1:3 to 1:20. The micronized drug has a particle size that is preferably smaller than 40 microns, even more preferably from 5 to 20 microns. The term collagen always makes reference to the partly hydroyzed industrial product thereof called gelatine. The collagen can be selected from among mammalian, chicken and fish collagen and, as said above, can be partly hydroyzed collagen having a Bloom degree ranging from 0 to 300, preferably from 50 to 300.

The drug (or pharmaceutical active ingredient) which forms part of the product of the invention can be any drug susceptible of amorphization. In general, the drug usable in the product according to the present invention is a solid crystalline compound that is soluble, insoluble or poorly soluble in water. Clearly, the advantage of rendering the drug more bioavailable according to the present invention will be all the more evident the more the amorphization renders the drug insoluble or poorly soluble in water, thereby facilitating the passage thereof through a lipophilic membrane. Among the drugs that can constitute part of the product of the invention it is possible to mention, solely by way of non-limiting example, solid crystalline nonsteroidal anti-inflammatory drugs, such as, for example, acetylsalicylic acid, ibuprofen and nimesulide, corticosteroids, such as, for example, dexamethasone, or antibiotics. According to one embodiment of the present invention, the drug is other than JM216.

The present invention further relates to a pharmaceutical composition comprising or consisting of the product as defined above, as an active ingredient, in association with one or more excipients and/or coadjuvants utilizable in pharmaceutical formulations.

The pharmaceutical composition according to the present invention can further comprise a drug (or pharmaceutical active ingredient) not co-ground with collagen in order to impart additional beneficial effects to the composition.

The pharmaceutical composition according to the present invention can be formulated for oral use in swallowable or orodispersible form, for example in the form of capsules, tablets and syrups, for sublingual use, for example in the form of tablets, drops and granules, for topical use on the skin or mucosa, for example in the form of powders to be sprinkled, gels, creams, mouthwashes, eye drops, sprays and suppositories, or for inhalatory use. The sublingual pharmaceutical composition comprising the product of the invention containing NSAIDs, such as, for example, acetylsalicylic acid, is particularly advantageous.

On the basis of what has been illustrated above, therefore, the product and pharmaceutical composition according to the present invention can be advantageously used in the medical field.

The present invention further relates to a process for the preparation of the product as defined above, said process comprising the steps of a) preparing a powder mixture of at least one crystalline micronized drug (or pharmaceutical active ingredient) and collagen; b) grinding the mixture of step a) until amorphization of said at least one crystalline micronized drug is obtained.

Co-grinding can be carried out in a mortar or any other crushing apparatus until achieving deconstruction of the crystalline structure of the drug.

The amorphization of the drug can be verified by means of an analytic method capable of detecting the deconstruction of the crystalline edifice, such as, for example, differential scanning calorimetry or optical microscopy or Raman spectroscopy.

According to the process of the present invention, the ratio by weight between said at least one drug and the collagen can range from 1:1 to 1:30, preferably from 1:3 to 1:20. Furthermore, the particle size of the drug is preferably smaller than 40 microns, even more preferably it can range from 5 to 20 microns.

As illustrated above, the collagen can be partly hydroyzed collagen, preferably with a Bloom degree that can range from 0 to 300, preferably from 50 to 300.

The invention will be described below by way of non-limiting illustration, with particular reference to several illustrative examples and the figures in the appended drawings, in which:

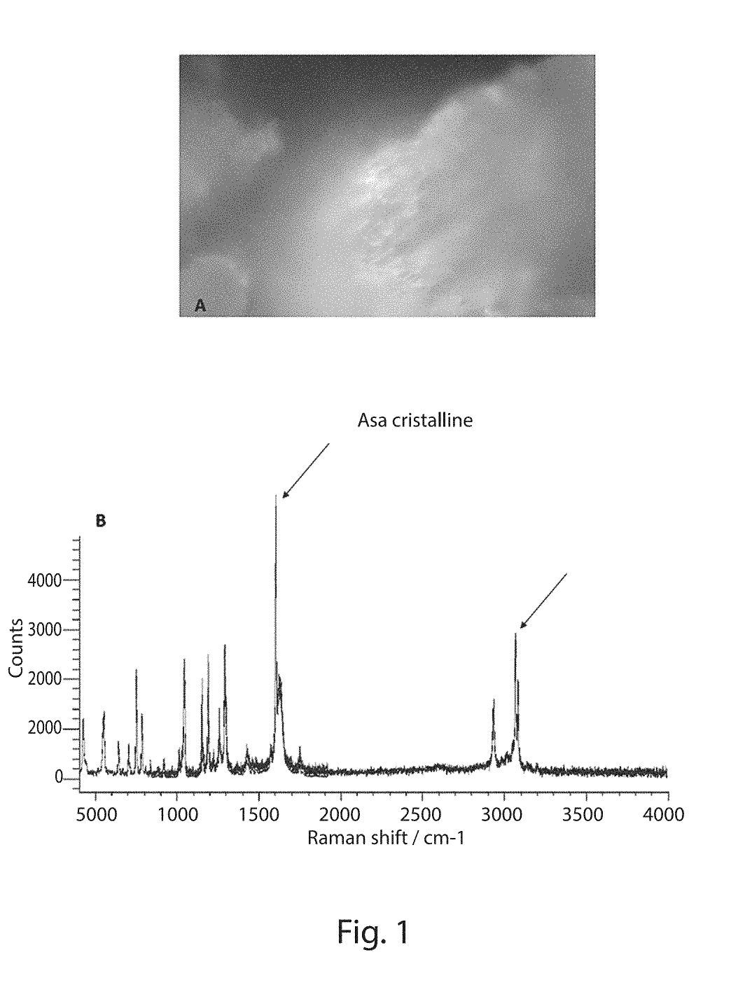

FIG. 1 shows a microscope image obtained with RAMAN technology. The microphotograph on the left of the photo shows an image of acetylsalicylic acid in crystalline form, a fact confirmed by the spectrum analysis (right part of the figure), which documents peaks of activity consistent with the crystalline structure.

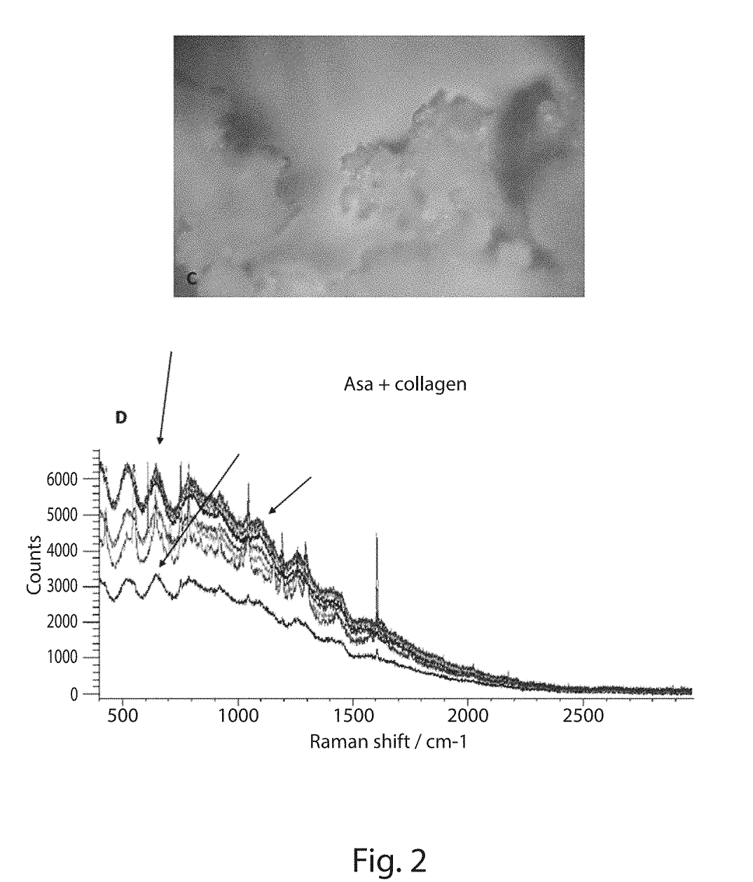

FIG. 2 shows how the micronization of acetylsalicylic acid and subsequent co-grinding of the drug with bovine collagen leads to a deconstruction of the crystalline form of the acetylsalicylic acid (part C of the figure) with amorphization documented by the Raman spectrum analysis in section D.

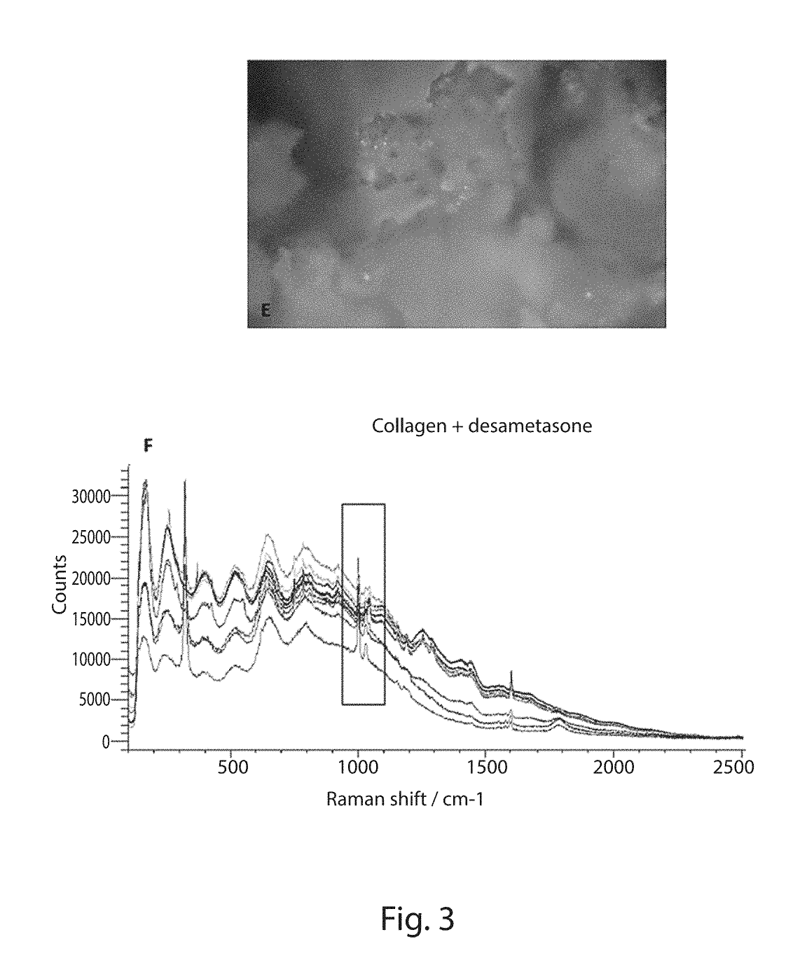

FIG. 3 shows, similarly to what is documented in FIG. 2, that dexamethasone is also rendered amorphous by micronization and subsequent co-grinding with collagen.

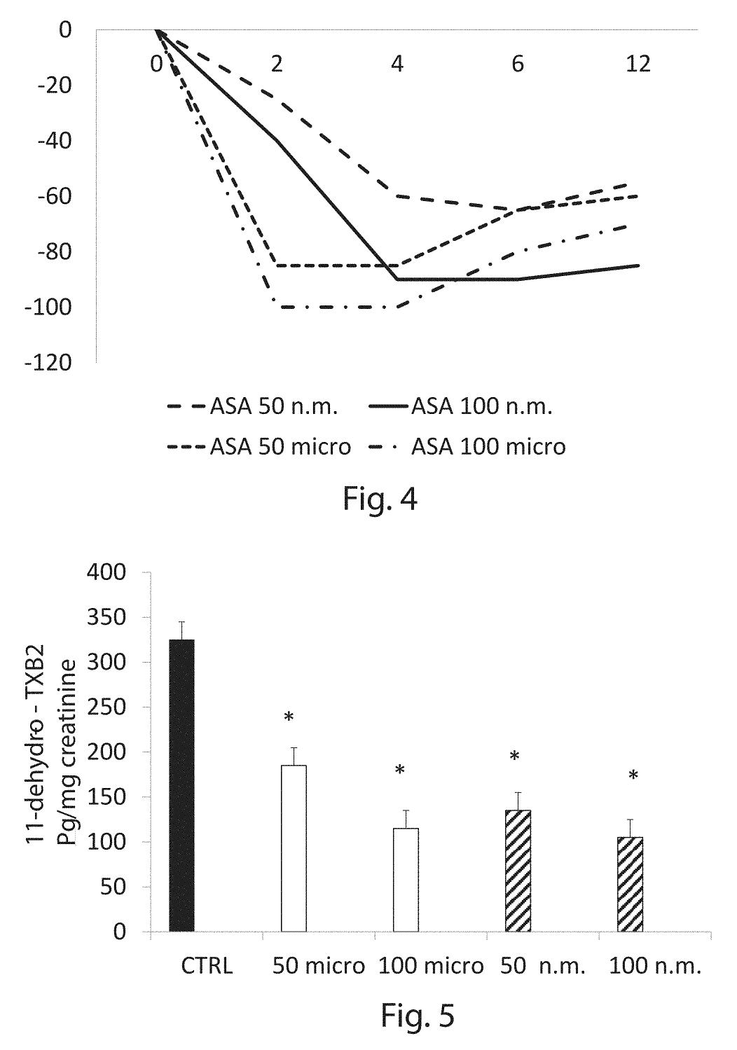

FIG. 4 shows the effects of micronization with collagen on the effect of acetylsalicylic acid (ASA) administered sublingually in healthy volunteers.

FIG. 5 shows the effects of micronization and co-grinding with collagen on the effect of ASA on the levels of urinary 11-dehydro-TXB2 after 7 days of treatment in healthy volunteers.

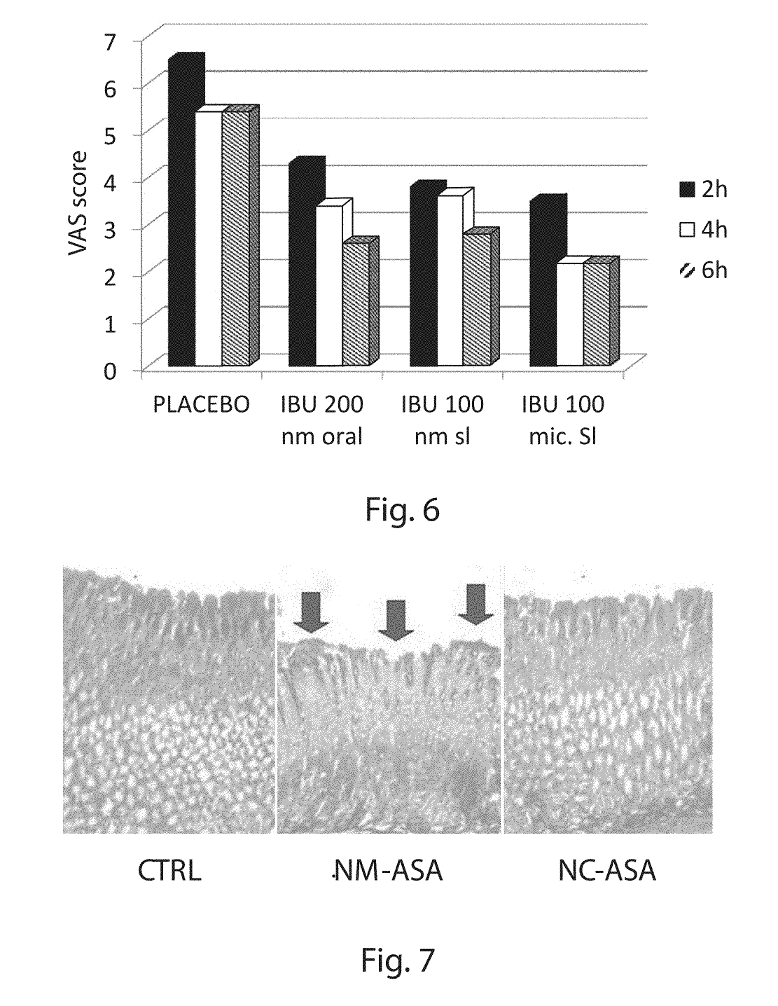

FIG. 6 shows the effects of ibuprofen (IBU) administered sublingually at a dose of 100 mg, micronized and mixed together with collagen (mic) or non-micronized (nm), and 200 mg of non-micronized ibuprofen given orally, on the score obtained on the visual analog scale (VAS) in patients with scapulohumeral periarthritis.

FIG. 7 shows how the product obtained by micronization of ASA (400 mg/Kg) and co-grinding of the same with collagen, administered orally, produces gastro-protective effects vis-à-vis non-micronized ASA.

### EXAMPLE 1

### Process of Preparation of a Product According to the Present Invention containing Amorphous Micronized Acetylsalicylic Acid (ASA) and Collagen

A mixture of collagen in the form of the partly hydroyzed industrial derivative thereof called gelatine and micronized acetylsalicylic acid (ASA) in a ratio of 3 to 1 was ground manually in a mortar. A check by optical microscopy at 30 minutes revealed the disappearance of the microcrystals, replaced by amorphous particles surrounded by collagen. This fact was confirmed by differential scanning calorimetry performed with a Perkin Elmer DSC 7 apparatus, calibrated with indium. The samples were examined with a scanning speed of 5.0 C/min. By way of comparison, three samples of ASA were used, a granular one (ASA code 3118) and two in powder form (ASA 3220 I and ASA 3220 II). All the samples were accompanied with a certificate of analysis. In the products identified as ASA 3118 (granular), ASA 3220 I and ASA 3220 II (both in powder form) the melting peak was observed in the interval of 133.9-136.8, which distinguishes the crystalline edifice. In the mixture of ASA 3220 I and hydrolyzed bovine collagen, prepared in a mortar in a ratio of 1 to 3, the melting peak between 135° C. and 138° C. was absent. In the control samples prepared with non-micronized ASA, the glue effect manifested itself before the completion of the amorphization process, preventing it.

The process of amorphization of the drug was demonstrated in an incontestable manner using Raman spectroscopy. In particular, with this method it was possible to document how the co-grinding of acetylsalicylic acid with collagen leads to a mixture in which ASA is amorphized and complexed with collagen, which assures the state of amorphization over time (FIGS. 1, 2).

The same result was obtained by co-grinding micronized dexamethasone and collagen (FIG. 3).

Therefore, the micronization and subsequent co-grinding of a drug can be extended to all drugs distinguished by a solid crystalline state, which can be soluble, insoluble or poorly soluble in water. Clearly, the advantage of rendering the drug more bioavailable according to the present invention will be all the more evident the more the amorphization renders the drug insoluble or poorly soluble in water, thereby facilitating the passage thereof through a lipophilic membrane. The result is the formation of a pharmaceutical composition that lends itself to tests of clinical effectiveness, as illustrated in the examples below.

### EXAMPLE 2

### Pharmaceutical Compositions Containing the Product of the Present Invention based on ASA and Collagen in the form of the Partly Hydroyzed Industrial Product thereof Called Gelatine

Pharmaceutical compositions based on ASA and collagen, in the ratio of 1 to 3, were prepared in a pharmacy in the form of the following preparations:

Capsules for oral use, each containing 150 mg of acetylsalicylic acid:

| | |
|---|---|
| Acetylsalicylic acid | 150 mg |
| Collagen 0 Bloom degree Magnesium stearate | 10 mg |
| Acetylsalicylic acid | 200 mg |
| Collagen 0 Bloom degree | 400 mg |
| Magnesium stearate | 10 mg |

Tablets for sublingual use, each containing 30 mg of acetylsalicylic acid

| | |
|---|---|
| Acetylsalicylic acid | 30 mg |
| Collagen 0 Bloom degree | 110 mg |
| Microcrystalline cellulose | 40 mg |
| Carboxymethyl amide-sodium | 10 mg |
| Amarena cherry flavouring | 6 mg |
| Sucralose | 5 mg |
| Magnesium stearate | 3 mg |

### EXAMPLE 3

### Study on Healthy Volunteers to Determine the Bioavailability and Effectiveness of Sublingual and Oral Compositions According to the present Invention Containing ASA and Collagen in the form of the Partly Hydroyzed Industrial Product thereof Called Gelatine

The pharmaceutical composition obtained by co-grinding a micronized drug with gelatine was tested in healthy volunteers in order to verify whether the product, in its amorphized form, brought advantages from the standpoint of bioavailability and an enhanced clinical effectiveness associated with an improvement in the safety profile. These tests confirmed that the co-grinding process and simultaneous amorphization of ASA with collagen enables a better absorption of the drug without compromising its effectiveness.

In particular, the administration of the pharmaceutical composition of ASA+collagen after co-grinding in tablet form at doses of 50 and 100 mg given sublingually and orally in groups of 20 healthy volunteers brought about an increase in the plasma concentrations of ASA, determined using the LC-MS method, compared to the administration of equivalent doses of ASA in crystalline form administered sublingually or orally. Table 1 shows the effects of micronization on the pharmacokinetic parameters of the ASA administered orally or sublingually.

TABLE 1

| | ASA 50 | | | | | ASA 100 |
|---|---|---|---|---|---|---|
| Pharmacokinetic | oral | ASA 100 | ASA 50 | ASA 100 | ASA 50 | Sublingual |
| parameters | n.m. | oral n.m. | Sublingual n.m. | Sublingual n.m. | Sublingual micro. | micro. |
| AUC (0-24 h) | 532.00 ± 123.260 | 1 028.00 ± 53.30 | 675.20 ± 75.56 | 1 128.00 ± 67.45 | 825.38 ± 85.20* | 1485.20 ± 77.40* |
| ng mL⁻¹ × h | | | | | | |
| Cmax ng mL−1 | 268.70 ± 85.40** | 459.90 ± 73.20** | 314.33 ± 89.50 | 542.81 ± 68.29 | 465.20 ± 91.41* | 685.39 ± 77.81* |

*P < 0.05 formulation of micronized vs non-micronized ASA 50 and 100 mg and collagen

This effect was accompanied by a considerable therapeutic effectiveness, given that the plasma peak of thromboxane B2, a biomarker of the anti-aggregation effectiveness of ASA, came 1.5 hours earlier than with the crystalline form of the drug (FIG. 4). Moreover, a better gastric tolerability of ASA was observed, to be attributed to the protective action of the ASA+collagen mixture on the wall. This fact was confirmed by examining both the plasma and urine concentrations of ASA and of the urinary metabolites of thromboxane after 7 days of administration (FIG. 5). It should be noted that collagen on its own produced no significant effects.

### EXAMPLE 4

### In Vivo Study on the Bioavailability and Effectiveness of Topical Compositions According to the Present Invention Containing Dexamethasone and Collagen in the Form of the Partly Hydroyzed Industrial Product thereof Called Gelatine

An increase in effectiveness compared to the drug applied in crystalline form was also documented with topical administrations of the pharmaceutical composition of the invention. In fact, in an experimental animal in whose paw 0.20 ml of a carrageenan solution was administered, the mixture derived by co-grinding dexamethasone+collagen produced a significantly larger reduction in carrageenan-induced edema and erythema than dexamethasone administered in its crystalline form. This effect, besides being quantitatively greater, was manifested earlier, confirming the better bioavailability of the pharmaceutical composition applied topically compared to the drug in on its own produced no effect.

### EXAMPLE 5

### Study on Subjects with Scapulohumeral Periarthritis to Determine the Bioavailability and Effectiveness of the Sublingual Compositions According to the Present Invention Containing Ibuprofen and Collagen in the form of the Partly Hydroyzed Industrial Product thereof Called Gelatine

The data relating to the effectiveness of the pharmaceutical composition were confirmed by experimental data obtained in three groups of subjects with scapulohumeral periarthritis.

Ibuprofen (IBU) was administered to patients with scapulohumeral periarthritis sublingually at a dose of 100 mg, micronized and mixed together with collagen (mic) or non-micronized (nm), and 200 mg of non-micronized ibuprofen given orally.

In these subjects, it was verified that the combination of micronized ibuprofen co-ground with collagen is the one that has the best profile of effectiveness both in terms of the threshold of the effect and the intensity of the pain-killing response (FIG. 6).

### EXAMPLE 6

### Study of the Effects of the Micronization of ASA and Co-Grinding with Collagen, in the form of the Partly Hydroyzed Industrial Product thereof Called Gelatine, on Gastroerosive Activity following Administration of the Drug on the Gastric Mucosa

It is well known that ASA, in its traditional crystalline form, is capable of causing gastroerosive effects, which manifest themselves in patients with alterations of the gastric mucosa of a varioliform type (gastritis), eventually resulting in the appearance of veritable ulcerations of the mucosa. This fact is only partly determined by effects of type 1 cyclooxygenase-inhibiting drugs (NSAIDs) on prostaglandin synthesis. These mediators, in fact, are responsible for gastroprotection under normal conditions, thanks to their effect on the production of gastric mucous, which opposes the lowering of the pH during the gastric phase of digestion. In reality, other factors contribute to the gastroerosive effect of NSAIDs, and of ASA in particular, including the “wall” effect connected with the direct irritating action of the drug toward the gastric mucosa.

In view of this, it was decided to test the effect of the micronization of ASA (400 mg/Kg) and co-grinding thereof with collagen through oral administration on the mucosa of Wistar rats weighing 210 g and compare it with the effects of the administration of ASA (400 mg/Kg) in its non-micronized form.

The effects are reported in Table 2 and in FIG. 7.

TABLE 2

| GROUP | Ulcer index |
|---|---|
| CTRL | No lesion |
| NM-ASA | 36.12 ± 4.2* |
| MC-ASA | 12.5 ± 3.9⁺⁺ |

*P < 0.05 NM-ASA vs control
⁺⁺P < 0.05 NM_ASA vs MC-ASA

In particular, a calculation was made of the effect of ASA on the so-called “ulcer score”, according to a method that differentiates the damage due to the acute administration of high doses of ASA given orally into classes of severity which range from the absence of lesions to the maximum score, which is equivalent to ulcers equal to or greater than 6 mm. Furthermore, an analysis was made of the effects of ASA, in the two formulations used, on the histopathological damage of the gastric mucosa after periodic acid Schiff/Alcian blue staining of the histological samples taken from the animals after the two treatments (FIG. 7).

The data reveal that, following the administration of oral ASA in its non-micronized form, a high ulcer score (36.12±4.2) is observed, whereas this value is greatly attenuated in the event of administration of micronized ASA co-ground with collagen (12.5±3.9). Moreover, the histopathological damage characterized by the degeneration of cells of the gastric mucosa, which occurs following the administration of non-micronized ASA, is significantly milder when the ASA is administered in its micronized form co-ground with collagen.

These data document that the micronization of ASA and the co-grinding thereof with collagen succeeds in imparting gastroprotective properties to the pharmaceutical composition, as well as implementing the absorption thereof. This finding can be extended to other NSAIDs and other hydrophobic and non-hydrophobic substances.

### REFERENCES

Baigent C, Blackwell L, Collins R et al. Aspirin in the primary and secondary prevention of vascular disease: collaborative meta-analysis of individual participant data from randomised trials. Lancet 2009; 373(9678):1849-1860

Barzegar-Jalalia M, Valizadeh H, Shadbada M-R S, Adibkiaa K, Mohammadia G, Farahania A, Arasha Z and Nokhodchi A. Cogrinding as an approach to enhance dissolution rate of a poorly water-soluble drug (gliclazide). Powder Technology. 2010; 197(3): 150-158

Carli F. Pharmaceutical composition. 1987, U.S. Pat. No. 4,639,370 A Carli F, lamartino P, Leone M. Drug/carrier inclusion composites prepared by a mechanochemical activation process using high-energy fluid-jet mills. WO 2013110789 A

Castro G A, Sgarbieri V C, Carvalho J E, Tinti S V, Possent, A. Protective effect of collagen derivates on the ulcerative lesions caused by oral administration of ethanol. Journal of Medicinal Food 2007; 10(1):154-158

Castro G A, Carvalho J E, Tinti S V, Possenti A, Sgarbieri V C. Anti-ulcerogenic effect of a whey protein isolate and collagen hydrolysates against ethanol ulcerative lesions on oral administration to rats. Journal of Medicinal Food. 2010; 13(1):83-90

Cioli V, Putzolu S, Rossi V, Scorza Barcellona P, Corradino C. The role of direct tissue contact in the production of gastrointestinal ulcers by anti-inflammatory drugs in rats. Toxicol Appl Pharmacol. 1979; 50(2):283-9

Cooper S A and Voelker M. Evaluation of onset of pain relief from micronized aspirin in a dental pain model. Inflammopharmacology, 2012, 20 (4): 233-42

Dingding Chen and Lizhong Gao. Medical and health-care uses of pufferfish Type the collagen extract and processes for producing said extract. 2007. U.S Pat. No. 2007219128 A1

García Rodríguez the and Jick H. Risk of upper gastrointestinal bleeding and perforation associated with Individual non-steroidal anti-inflammatory drugs. The Lancet 1994, 343(8900: 769-772

Gareis H and Schrieber R. Gelatine Handbook: Theory and Industrial Practice. Weinheim: Wiley-VCH, 2007

Gohel M C and Patel D. Improvement of Nimesulide Dissolution by a Co-grinding Method Using Surfactants. Pharmacy and Pharmacology Communications. 2000, 6(10): 433-440

Guangli Mu and Xingfu Ma. Casing smooth muscle collagen preparation method and functional study. Intestine casing smooth muscle collagen, its preparation method and application. CN Patent No.102020711 A

Gupta M K, Vanwert A, Bogner R H, Formation of physically stable amorphous drugs by milling with Neusilin. J Pharm Sci 2003, 92: 502-517

Huang E S, Strate L L, Ho W W, Lee S S, Chan A T, Long Term Use of Aspirin and the Risk of Gastrointestinal Bleeding. Am J Med 2011 124(5): 426-433

Joshi, J T. A review of Micronization. J. Pharm. Sci.Tech. 2011, 3(7): 651-681

Newman A, Knip G and Zografi G. Assessing the performance of amorphous solid dispersions. J Pharm Sci, 2012, (4): 1355-77

Newman A, Nagapudi K, Wenslow R. Amorphous solid dispersions: a robust platform to address bioavailability challenges. Ther Deliv. 2015, 6(2):247-61

Schreiber R and Gareis, H. Gelatine Handbook: Theory and Industrial Practice. Weinheim: Wiley-VCH, 2007

Serajuddin Abu T M. Solid dispersion of poorly water-soluble drugs: Early promises, subsequent problems, and recent breakthroughs. Journal of Pharmaceutical Sciences, 1999, 88 (10), 1058-1066

Silvestrini B. A novel composition, comprising tricalcium phosphate and gelatin, for treating dyspepsia and related disorders. 2015/15425001

Silvestrini B and Bonanomi M. Administration sublinguale of antinfiammatori non steroidei. IT 1343576 (2008)

Silvestrini B and Bonanomi M. Composition pharmaceutical per the administration sublinguale of a sostanza dotata of attivita antiaggregante piastrinica. IT 1357717 (2009)

Silvestrini B and Bonanomi M. Administration sublinguale of antinfiammatori non steroidei, IT 1343575 (2008)

Silvestrini B and Bonanomi M. Use of partly hydrolyzed fish gelatin for the preparation of topically administered pharmaceutical compositions. WO 2009/083768 A2

Sreenivasa R K S, Shanelle W, Rupa S, Sarah N, Sebhat E, Naveed S, Kausik K R. Vascular benefits are offset by clinically important bleeding events. Arch Intern Med. Published online Jan. 9, 2012

US Preventive Services Task Force. Aspirin for the prevention of cardiovascular disease: U.S. Preventive Services Task Force recommendation statement. Ann. Intern. Med. 2009; 150(6):396-404

Vadher A H, Parikh J R, Parikh R H and Solanki A B. Preparation and characterization of co-grinded mixtures of aceclofenac and neusilin US2 for dissolution enhancement of aceclofenac. PharmSciTech. 2009; 10(2):606-14

Voelker M and Hammer M. Dissolution and pharmacokinetics of a novel micronized aspirin formulation. Inflammopharmacology, 2012, 20 (4): 225-31

Watanabe T, Ohnoa I, Wakiyamaa N, Kusaia A, Sennab M. Stabilization of amorphous indomethacin by co-grinding in a ternary mixture. International Journal of Pharmaceutics, 2002, 241(1):103-111

Wongmekiat A, Yoshimatsu S, Tozuka Y, Moribe K, Yamamoto K. Investigation of Drug Nanoparticle Formation by Co-grinding with Cyclodextrins: Studies for Indomethacin, Furosemide and Naproxen. Journal of inclusion phenomena and macrocyclic chemistry, 2006, 56 (1): 29-32

Zhou Xin, Han Yang and Xu Jianwen. Functional composition for improving gastrointestinal function, supplementing collagen as well as protecting skin, bones, joints and blood vessels. 2011, CN Patent No.102132814 A

What is claimed is:

1) Product, or composition product, comprising or consisting of at least one amorphous micronized drug and collagen, said product being obtainable through amorphization by co-grinding at least one crystalline micronized drug in powder mixture with collagen.

2) Product according to claim 1, wherein the ratio between said drug and collagen ranges from 1:1 to 1:30 by weight, preferably from 1:3 to 1:20.

3) Product according to anyone of claims 1-2, wherein said at least one drug has particle size smaller than 40 microns, preferably from 5 to 20 micron.

4) Product according to anyone of claims 1-2, wherein collagen has Bloom degree from 50 to 300.

5) Product according to anyone of claims 1-2, wherein said at least one drug is chosen from the group consisting of solid crystalline nonsteroidal anti-inflammatory drugs, such as acetylsalicylic acid, ibuprofen and nimesulide, corticosteroid drugs such as dexamethasone, or antibiotics.

6) Pharmaceutical composition comprising or consisting of the product as defined in anyone of claims 1-5, as active principle, in association with one or more excipients and /or coadjuvant.

7) Pharmaceutical composition according to claim 6, said pharmaceutical composition further comprising a drug not co-ground with collagen.

8) Pharmaceutical composition according to anyone of claims 6-7, wherein said pharmaceutical composition has a form chosen among swallowable or orodispersible oral form, sublingual form, topical form for skin or mucosa, and inhalatory form.

9) Product as defined in anyone of claims 1-5 or pharmaceutical composition as defined in anyone of claims 6-8, for use in the medical field.

10) Process for the preparation of the product as defined in anyone of claims 1-5, said process comprising the steps a) preparing a powder mixture of at least one crystalline micronized drug and collagen; b) grinding the mixture of step a) until amorphization of said at least one crystalline micronized drug is obtained.

11) Process according to claim 10, wherein the ratio between said at least one drug and collagen ranges from 1:1 to 1:30 by weight, preferably from 1:3 to 1:20.

12) Process according to anyone of claims 10-11, wherein said at least one drug has particle size smaller than 40 microns, preferably from 5 to 20 microns.

13) Process according to anyone of claims 10-11, wherein the collagen has Bloom degree from 50 to 300.
